(19) United States
(12) Patent Application Publication
(10) Pub. No.: US 2004/0180084 A1
(43) Pub. Date: Sep. 16, 2004

(54) Composition for the treatment and prevention of cardiovascular disease, stroke, alzheimer's disease and neurological degeneration

(76) Inventor: Wayne F. Gorsek, Boynton Beach, FL (US)

Correspondence Address: Hoffman, Wasson & Gitler, P.C., Suite 522, 2361 Jefferson Davis Highway, Arlington, VA, 22202, US

(21) Appl. No.: 10/385,635

(22) Filed: Mar. 12, 2003

(51) Int. Cl. A61K 31/714; A61K 31/525; A61K 31/315; A61K 31/198; A61K 31/045

(52) U.S. Cl. 424/465; 514/251; 514/52; 514/494; 514/562; 514/729; 514/89; 514/350

(57) ABSTRACT

The invention relates to a composition that contains the most potent combination of nutrients proven to assist in the treatment and prevention of cardiovascular disease, stroke, Alzheimer's disease and neurological degeneration by lowering elevated homocysteine levels. The present invention involves a composition for oral ingestion that contains essential amounts of Vitamin B6(Pyroxidine HCL and Pyridoxal 5-Phosphate), Folic Acid(folacin), Vitamin B12(Methylcobalamin), Zinc(Zinc L-monomethionine), N-acetyl Cysteine, and Inositol, as well as other ingredients and healthy filler ingredients.

### BACKGROUND OF THE INVENTION

The invention relates to a composition that contains the most potent combination of nutrients with clinical studies proven to assist in the treatment and prevention of cardiovascular disease, stroke, Alzheimer's disease and neurological degeneration.

The advanced formulation is designed to prevent elevated homocysteine levels and lower high levels of homocysteine to optimized desired levels. High homocysteine levels cause damage to both arteries and veins and prolonged elevations lead to an increase risk of blood clotting along with interference in the way blood vessels dilate. Elevated levels of homocysteine can lead to the development of Alzheimer's disease and other forms of dementia.

Emerging research confirms that nutrition may play a significant role in assisting the body to repair the damage to arteries and veins. Vitamin B6 (Pyroxidine HCL and Pyroxidal-5-Phosphate or other forms), Folic Acid, Vitamin B12(Methylcobalamin or other forms), N-Acetyl-Cysteine, Inositol, and Zinc have been shown to actually lower the levels of homocysteine. In addition to their effectiveness, all of these dietary supplements are extremely safe and free of dangerous side effects.

The best, the easiest and most cost-effective way to give your veins and arteries the nutritional support they need is with a high-quality product that combines all the right supplements in the right dosages as embodied by the inventive formulation.

It is an object of the present invention to provide an unique and superior formulation which allows individuals to reduce and lower homocysteine levels in the body to a healthy or optimal level. This will prevent or treat cardiovascular disease, stroke, Alzheimer's disease and neurological degeneration.

### SUMMARY OF THE INVENTION

The key to the unique formulation is a combination of specific vitamins, and other necessary nutrients. These essential components in the amounts provided uniquely reduce and lower homocysteine levels in the body to a healthy or optimal level.

The formulation contains essential amounts of Vitamin B6(Pyroxidine HCL and pyridoxal 5-phosphate), Folic Acid(folacin), Vitamin B12(Methylcobalamin), Zinc(Zinc L-monomethionine), N-acetyl Cysteine and Inositol.

The formulation is preferably delivered in capsule form at 1 or 2 capsules per day.

### DETAILED DESCRIPTION OF THE INVENTION

The present invention involves a composition for oral ingestion that contains essential amounts of Vitamin BG(Pyroxidine HCL and Pyridoxal 5-Phosphate), Folic Acid(folacin), Vitamin B12(Methylcobalamin), Zinc(Zinc L-monomethionine), N-acetyl Cysteine, and Inositol, as well as other ingredients and healthy filler ingredients. More specifically, this formulated product is a homocysteine level maintenance and management formulation. This formulation allows for the treatment and prevention of cardiovascular disease, stroke, Alzheimer's disease and neurological degeneration.

In order to secure the desired result the following essential components are provided:

Vitamin B6, preferably in the form of pyridoxine HCL and Pyridoxal 5-Phosphate.

Folic acid, preferably in the form of Folacin.

Vitamin B12, preferably in the form of methylcobalamin.

Zinc in the form of Zinc L-monomethionine.

N-acetyl Cysteine(NAC), a thiol-containing compound interfering with endogenous thiols, cysteine and plasma homocysteine(Hcy), by forming with them mixed disulphides with a possibly more efficient renal clearance.

Insitol

Additionally, the following is a breakdown of the preferred formulation:

| | |
|---|---|
| Vitamin B6 (pyroxidine HCL) | 50 mg |
| | (5-10,000 mg) |
| Folic Acid(folacin) | 2 mg |
| | (200 mcg-40 mg) |
| Vitamin B12(methylcobalamin) | 2 mg |
| | (10 mcg-40 mg) |
| Zinc(Zinc L-monomethionine) | 15 mg |
| | (1-300 mg) |
| Vitamin B6 (Pyroxidal 5-Phosphate) | 50 mg |
| | (5-5,000 mg) |
| N-Acetyl Cysteine | 300 mg |
| | (30-6,000 mg) |
| Inositol | 125 mg |
| | (10-5000 mg) |

(other ingredients: Cellulose, Magnesium Stearate, and Kosher Gelatin Capsule)

In addition to the key components, other components such as kosher gelatin (capsules), magnesium stearate and cellulose are included.

The formulations described help maintain healthy homocysteine levels for life. Although the invention has been described primarily in connection with special and preferred embodiments, it will be understood that it is capable of modification without departing from the scope of the invention. The following claims are intended to cover all variations, uses, or adaptations of the of the invention, following, in general the principles thereof and including such departures from the present disclosure as come within known or customary practice in the field to which the invention pertains, or as are obvious to persons skilled in the field.

What is claimed is:

1. A composition for lowering and maintaining homocysteine levels and for the treatment and prevention of cardiovascular disease, stroke, Alzheimer's disease and neurological degeneration comprising an effective amount of:
Vitamin B6 (pyroxidine HCL and Pyroxidal 5-Phosphate)
Polic Acid(folacin)
Vitamin B12(methylcobalamin)
Zinc(Zinc L-monomethionine)
N-Acetyl Cysteine; and
Inositol.

2. A composition for lowering and maintaining homocysteine levels and for the treatment and prevention of cardiovascular disease, stroke, Alzheimer's disease and neurological degeneration comprising an effective amount of:
5 to 5,000 mg Vitamin B6 (pyroxidine HCL and Pyroxidal 5-Phosphate)
200 mcg-40 mg Folic Acid(folacin)
10 mcg-40 mg Vitamin B12(methylcobalamin)
1-300 mg Zinc(Zinc L-monomethionine)
30 to 6,000 mg N-Acetyl Cysteine; and
10 to 5,000 mg Inositol.
